(19) United States
(12) Patent Application Publication
(10) Pub. No.: US 2024/0026462 A1
(43) Pub. Date: Jan. 25, 2024

(54) HOMOLOGOUS RECOMBINATION DEFICIENCY DETERMINING METHOD AND KIT THEREOF

(71) Applicants: Ying Ja CHEN, Taipei City (TW); Shu Jen CHEN, Taipei City (TW); ACT GENOMICS (IP) LIMITED, Hong Kong City (HK)

(72) Inventors: WOEI-FUH WANG, Taipei City (TW); YA-CHI YEH, Taipei City (TW); YING-JA CHEN, Taipei City (TW); SHU-JEN CHEN, Taipei City (TW); CHIEN-HUNG CHEN, Taipei City (TW); KUAN-YING CHEN, Taipei City (TW); WEN-HAO TAN, Taipei City (TW)

(21) Appl. No.: 18/346,875

(22) PCT Filed: Aug. 13, 2021
(86) PCT No.: PCT/US2021/046034

§ 371 Date: Jul. 5, 2023

Related U.S. Application Data
(60) Provisional application No. 63/135,622, filed on Jan. 10, 2021.

(51) Int. Cl. C12Q 1/6886; G16B 20/20

(52) U.S. Cl. CPC C12Q 1/6886; G16B 20/20; C12Q 2600/156

(57) ABSTRACT

The present disclosure provides a method, a system and a kit for assessing the homologous recombination deficiency (HRD) status of a subject. The present disclosure further provides a method, a system and a kit for identifying a treatment based on the HRD status for the human subject.

### CROSS-REFERENCE TO RELATED APPLICATIONS

This application claims priority of Provisional Application No. 63/135,622, filed on Jan. 10, 2021, the content of which is incorporated herein in its entirety by reference.

### FIELD

The disclosure relates to a method and a kit for assessing homologous recombination deficiency (HRD) status.

### BACKGROUND OF THE INVENTION

Poly (ADP-ribose) polymerases (PARPs) pathway and homologous recombination repair (HRR) pathway are involved in DNA damage repair. Inhibition of PARP may cause unrepaired DNA single-strand breaks (SSBs) and stalled replication forks accumulate, resulting in collapse of replication forks and generation of double-strand DNA breaks (DSBs) during DNA replication, which are repaired by HRR pathway in normal cells. When HRR is deficient, synthetic lethality occurs in the presence of PARP inhibition. Nowadays, PARP inhibitor has been developed as a cancer drug for treating patients with homologous recombination deficiency (HRD).

For PARP inhibitor treatment, biomarker testing (i.e., BRCA1/2 mutation status) is mostly required prior to PARP inhibitor treatment initiation to identify the patients that would most benefit from the therapy. So far, only two companion diagnostic tests, Myriad myChoice and FoundationFocus, for PARP inhibitor treatment have been approved by FDA. There is still a need for developing more companion diagnostic assays to determine the HRD status for patients.

### SUMMARY OF THE INVENTION

In one general aspect, the disclosure relates to a method for assessing homologous recombination deficiency (HRD) status in a subject, including:
(1) sequencing multiple single nucleotide polymorphism (SNP) loci of a sample from the subject, wherein at least 50% of interval between every two neighboring SNP loci is 0.01 to 1 Mb in length;
(2) identifying number of loss of heterozygosity (LOH) SNP loci and number of non-homozygous SNP loci by sequencing result;
(3) calculating a LOH score, wherein the LOH score is a ratio of the number of LOH SNP loci to the number of non-homozygous SNP loci; and
(4) identifying HRD status by the LOH score.

In some embodiments, SNP loci is at least 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, or 300000 loci in number. In some embodiments, the SNP loci is 1000 to 260000, 2000 to 200000, 3000 to 100000, 3000 to 60000, 6000 to 11000, 7000 to 10000, or 7500 to 9500 in number. In some embodiments, the SNP loci are in at least 1, 2, 3, 4, 5, 6, 7, 8, 9, 10, 11, 12, 13, 14, 15, 16, 17, 18, 19, 20, 21, or 22 pairs of human chromosomes. In some embodiments, the SNP loci are in autosomal chromosomes. In some embodiments, the SNP loci are at 1p, 2p, 3p, 4p, 5p, 6p, 7p, 8p, 9p, 10p, 11p, 12p, 16p, 17p, 18p, 19p, 20p, 21p, 22p, 1q, 2q, 3q, 4q, 5q, 6q, 7q, 8q, 9q, 10q, 11q, 12q, 13q, 14q, 15q, 16q, 17q, 18q, 19q, 20q, 21q, and/or 22q of human chromosomal arms. In some embodiments, at least 50%, 55%, 60%, 65%, 70%, 75%, 80%, 85%, 90%, 95%, or 100% of intervals between the SNP loci is 0.01 to 3 Mb, 0.02 to 2 Mb, 0.03 to 1 Mb, 0.06 to 1 Mb, 0.1 to 1 Mb, 0.1 to 0.5 Mb, or 0.06 to 0.6 Mb in length. In some embodiments, the mean of intervals between the SNP loci is 0.01 to 3 Mb, 0.02 to 2 Mb, 0.03 to 1 Mb, 0.06 to 1 Mb, 0.1 to 1 Mb, 0.06 to 0.6 Mb, 0.1 to 0.5 Mb, or 0.2 to 0.4 Mb in length.

In some embodiments, the chromosomal aberration is loss of heterozygosity (LOH). In some embodiments, the HRD score is the LOH score. In some embodiments, the LOH score is a ratio of the number of non-homozygous SNP loci with the chromosomal aberration to the number of non-homozygous SNP loci. In some embodiments, the LOH score is a ratio of the number of LOH SNP loci to the number of non-homozygous SNP loci. In some embodiments, the non-homozygous SNP loci include the heterozygous SNP loci and LOH SNP loci. In some embodiments, the heterozygous SNP loci are identified from the SNP loci.

In some embodiments, the LOH score is adjusted through eliminating imbalanced chromosome arms. In some embodiments, the LOH score is a ratio of the number of LOH SNP loci in non-imbalanced chromosome arms to the number of the non-homozygous SNP loci in non-imbalanced chromosome arms. In some embodiments, the imbalanced chromosome arm is characterized by a predetermined ratio of the number of LOH SNP loci to the number of the non-homozygous SNP loci in a chromosome arm, wherein the predetermined ratio is at least 70%, 75%, 80%, 85%, 90%, 95%, or 100%.

In some embodiments, the ratio of the non-homozygous SNP loci with LOH for characterizing the imbalanced chromosome arm is adjusted based on the value of tumor purity of the sample. In some embodiments, the ratio of the non-homozygous SNP loci with LOH for identifying imbalanced chromosome arm is at least 70%, 75%, 80%, 85%, 90%, 95%, 98%, or 100%. In some embodiments, the value of the tumor purity is between 30% to 95% or 30% to 70%. In some embodiments, the value of the tumor purity is 30%, 35%, 40%, 45%, 50%, 55%, 60%, 65%, 70%, 75%, 80%, 85%, 90%, or 95%.

In some embodiments, the HRD status is identified as positive or negative. In some embodiments, a cutoff value of the LOH score for identifying HRD status is 0.3, 0.35, 0.4, 0.45, 0.5, 0.55, or 0.6.

In one general aspect, the disclosure relates to a method for assessing HRD status in a subject, including:
(1) sequencing the SNP loci of the sample from the subject, wherein there is an interval between every two neighboring SNP loci and at least 50% of the interval is 0.01 to 1 Mb in length;
(2) calculating the ratio of the number of LOH SNP loci to the number of non-homozygous SNP loci;
(3) identifying the HRD status.

In one general aspect, the invention relates to a method for assessing HRD status in a subject, comprising:
(1) sequencing at least one HRR-associated gene of the sample from the subject;
(2) determining whether any of the HRR-associated genes harbors an alteration;
(3) identifying the HRD status of the subject.

In some embodiments, the HRD status is identified as positive when at least one of the gene harbors an alteration. In some embodiments, the HRD status is identified as negative when none of the gene harbors an alteration.

In some embodiments, the alteration is selected from the group consisting of single nucleotide variant (SNV), insertion, deletion, amplification, gene fusion, and rearrangement. In some embodiments, the alteration is selected from the group consisting of SNV, small insertions and deletion (INDEL), large genomic rearrangement (LGR), and copy number variation (CNV). In some embodiments, the alteration is a germline alteration or a somatic alteration.

In one general aspect, the invention relates to a method for assessing HRD status in a subject, including:
(1) sequencing the genes including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof;
(2) determining whether any of the BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes harbors an alteration;
(3) identifying the HRD status.

In some embodiments, the method further comprises a step of identifying a treatment based on the HRD status for the subject and/or administering a therapeutically effective amount of a treatment to the subject.

In some embodiments, the treatment includes administering the drug including but not limited to a DNA damaging agent, an anthracycline, a topoisomerase I inhibitor, radiation, and/or a PARP inhibitor or any combination thereof. In some embodiments, the PARP inhibitor includes but not limited to olaparib, niraparib, rucaparib, and talazoparib.

In some embodiments, the method for assessing HRD status in a sample is implemented on a next-generation sequencing (NGS) computing platform. In some embodiments, the sample is sequenced by NGS assay. In some embodiments, the NGS system used in the NGS assay is including but not limited to the MiSeq, HiSeq, MiniSeq, iSeq, NextSeq and NovaSeq sequencers manufactured by Illumina, Inc., Ion Personal Genome Machine (PGM), Ion Proton, Ion S5 series and Ion GeneStudio S5 series manufactured by Life Technologies, Inc., BGlseq series, DNBseq series and MGIseq series, manufactured by BGI, and MinION/PromethION sequencers manufactured by Oxford Nanopore Technologies.

In some embodiments, the sequencing reads are generated from nucleic acids that are amplified from the original sample or the nucleic acids captured by the bait. In some embodiments, the sequencing reads are generated from a sequencer that required the addition of an adapter sequence. In some embodiments, the sequencing reads are generated from a method includes but not limited to hybrid capture, primer extension target enrichment, a molecular inversion probe-based method, or multiplex target-specific PCR.

In some embodiments, the sample originates from cell line, biopsy, primary tissue, frozen tissue, formalin-fixed paraffin-embedded (FFPE), liquid biopsy, blood, serum, plasma, buffy coat, body fluid, visceral fluid, ascites, paracentesis, cerebrospinal fluid, saliva, urine, tears, seminal fluid, vaginal fluid, aspirate, lavage, buccal swab, peripheral blood mononuclear cells (PBMC), circulating tumor cell (CTC), cell-free DNA (cfDNA), circulating tumor DNA (ctDNA), DNA, nucleic acid, purified nucleic acid, or purified DNA.

In some embodiments, the sample originates from a human subject. In some embodiments, the sample is a clinical sample. In some embodiments, the sample originates from a diseased patient. In some embodiments, the sample originates from a patient having cancer, solid tumor, or hematologic malignancy. In some embodiments, the sample originates from a patient having ovarian cancer, prostate cancer, breast cancer, or pancreatic cancer. In some embodiments, the sample originates from a patient having brain cancer, breast cancer, colon cancer, endocrine gland cancer, esophageal cancer, female reproductive organ cancer, head and neck cancer, hepatobiliary system cancer, kidney cancer, lung cancer, mesenchymal cell neoplasm, prostate cancer, skin cancer, stomach cancer, tumor of exocrine pancreas, or urinary system cancer. In some embodiments, the sample originates from a pregnant woman, a child, an adolescent, an elder, or an adult. In some embodiments, the sample is a research sample.

In some embodiments, the method further includes a step of outputting the HRD status to an electronic storage medium or a display.

In one general aspect, the disclosure relates to a method for assessing HRD status in a subject implemented on a NGS computing platform, including:
(1) assaying an alteration of the genes of the sample from the subject, including:
   (1a) sequencing the genes including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L genes or any combination thereof;
   (1b) determining whether any of the BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes harbors an alteration;
(2) calculating a HRD score of the sample, including:
   (2a) sequencing a plurality of single nucleotide polymorphism (SNP) loci of the sample;
   (2b) calculating the HRD score of chromosomal aberration;
(3) identifying the HRD status.

In one general aspect, the invention relates to a method for assessing HRD status in a subject implemented on a NGS computing platform, including:
(1) assaying an alteration of a plurality of genes in the sample from the subject, comprising:
   (1a) sequencing at least one HRR-associated gene;
   (1b) determining whether any of the HRR-associated genes harbors an alteration;
(2) calculating a LOH score in the sample, including:
   (2a) sequencing a plurality of SNP loci of the sample, wherein there is an interval between every two neighboring SNP loci and at least 50% of the interval are 0.01 to 1 Mb in length;
   (2b) calculating the ratio of the number of LOH SNP loci to the number of non-homozygous SNP loci;
(3) identifying the HRD status.

In some embodiments, the HRD status is identified as positive either at least one of the gene harbors an alteration or the score (i.e., the LOH score or the HRD score) is greater than a cutoff value.

In another general aspect, the invention relates to a system for assessing HRD status, and the system comprises a data storage device storing instructions for determining characteristics of HRD status and a processor configured to execute the instructions to perform a method including:
(1) sequencing a plurality of single nucleotide polymorphism (SNP) loci of the sample, wherein there is an interval between every two neighboring SNP loci and at least 50% of the interval are 0.01 to 1 Mb in length;
(2) calculating a loss of heterozygosity (LOH) score, wherein the LOH score is a ratio of the number of LOH SNP loci to the number of non-homozygous SNP loci;
(3) identifying HRD status.

In another general aspect, the invention relates to a system for assessing HRD status, and the system includes a data storage device storing instructions for determining characteristics of HRD status and a processor configured to execute the instructions to perform a method including:
(1) sequencing the genes including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L genes or any combination thereof;
(2) determining whether any of the BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes harbors an alteration;
(3) identifying HRD status.

In another general aspect, the invention relates to a system for assessing HRD status, and the system comprises a data storage device storing instructions for determining characteristics of HRD status and a processor configured to execute the instructions to perform a method including:
(1) assaying an alteration of a plurality of genes in the sample, including:
(1a) sequencing the genes including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L genes or any combination thereof;
(1b) determining whether any of the BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes harbors an alteration;
(2) calculating a HRD score in the sample, including:
(2a) sequencing single nucleotide polymorphism (SNP) loci of the sample;
(2b) calculating a HRD score of chromosomal aberration;
(3) identifying HRD status.

In another general aspect, the invention relates to a system for assessing HRD status, and the system comprises a data storage device storing instructions for determining characteristics of HRD status and a processor configured to execute the instructions to perform a method including:
(1) assaying an alteration of a plurality of genes in the sample, including:
(1a) sequencing at least one HRR-associated gene;
(1b) determining whether any of the HRR-associated genes harbors an alteration;
(2) calculating a LOH score in the sample, including:
(2a) sequencing a plurality of SNP loci of the sample, wherein there is an interval between every two neighboring SNP loci and at least 50% of the interval are 0.01 to 1 Mb in length;
(2b) calculating the ratio of number of LOH SNP loci to number of non-homozygous SNP loci;
(3) identifying HRD status.

In another general aspect, the invention relates to a kit for assessing HRD status in a sample, including:
(1) a set of oligonucleotides targeting a plurality of SNP loci;
(2) a set of oligonucleotides targeting a plurality of HRR-associated genes; and
(3) a computer program including instructions for executing a method for determining HRD status.

In another general aspect, the invention relates to a kit for assessing HRD status in a sample, including:
(1) a reagent, including:
a set of oligonucleotides targeting a plurality of SNP loci, wherein there is an interval between every two neighboring SNP loci and at least 50% of the interval are 0.01 to 1 Mb in length;
(2) a computer program, including:
instructions to calculate a LOH score, wherein the LOH score is a ratio of the number of LOH SNP loci to the number of non-homozygous SNP loci; and instructions to identify HRD status.

In another general aspect, the invention relates to a kit for assessing HRD status in a sample, including:
(1) a reagent, including:
a set of oligonucleotides targeting the genes including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L genes or any combination thereof;
(2) a computer program, including:
instructions to determine whether any of the BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes harbors an alteration; and
instructions to identify HRD status.

In another general aspect, the invention relates to a kit for assessing HRD status in a sample, including:
(1) a reagent, including:
a set of oligonucleotides targeting a plurality of SNP loci, wherein there is an interval between every two neighboring SNP loci and at least 50% of the interval are 0.01 to 1 Mb in length; and
a set of oligonucleotides targeting at least one HRR-associated gene;
(2) a computer program, including:
instructions to calculate a LOH score, wherein the LOH score is a ratio of the number of LOH SNP loci to the number of non-homozygous SNP loci; and identifying HRD status;
instructions to determine whether any of the HRR-associated genes harbors an alteration; and
instructions to identify HRD status.

In another general aspect, the invention relates to a kit for assessing HRD status in a sample, including:
(1) a reagent, including:
a set of oligonucleotides targeting a plurality of SNP loci of the sample; and a set of oligonucleotides targeting the genes including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L genes or any combination thereof;
(2) a computer program, including:
instructions to calculate a HRD score of chromosomal aberration; instructions to determine whether any of the BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes harbors an alteration; and instructions to identify HRD status.

In some embodiments, the computer program further includes instructions to identify a treatment based on the HRD status for the subject.

### BRIEF DESCRIPTION OF THE DRAWINGS

FIG. 1A-1D show stability of LOH score of different algorithms at a different number of SNP loci of two tumor samples (GSM956523 and GSM956527) and two normal samples (GSM956582 and GSM956597).

FIG. 2A shows the LOH score of different tumor groups and normal sample groups using Equation 1 at a different number of SNP loci.

FIG. 2B shows the LOH score of different tumor groups and normal sample groups using Equation 2 at a different number of SNP loci.

FIG. 3 shows LOH score at different tumor purity levels considering or ignoring chromosome arm imbalance factor.

FIG. 4 is a graph plotting length of the interval between the SNP loci.

FIG. 5 is a graph plotting the LOH distribution of different groups.

### DETAILED DESCRIPTION

Unless defined otherwise, all technical and scientific terms used herein have the same meaning as commonly understood by a person skilled in the art to which this disclosure belongs. As used herein, the singular forms “a”, “an”, and “the” include plural referents unless the context clearly dictates otherwise.

As used herein, “HRR-associated gene” refers to an HRR gene or a regulator or a modulator thereof. The alteration of the HRR-associated gene may cause the presence of HRD. In some embodiments, the HRR-associated gene is selected from the group consisting of BRCA1, BRCA2, ARID1A, ATM, ATR, ATRX, ABL1, BAP1, BARD1, BLM, BRIP1, CDK12, CHEK1, CHEK2, ERCC1, ERCC3, ERCC4, FANCA, FANCC, FANCD2, FANCE, FANCF, FANCG, FANCL, LIG3, MRE11, MSH2, MSH6, MLH1, NBN, PALB2, PTEN, PARP1, POLB, RAD50, RAD51, RAD51B, RAD51C, RAD51D, RAD52, RAD54L, UBE2A, XRCC2, DNMT3A, IDH1, IDH2, STAG2, and TP53 genes. In some embodiments, the HRR-associated gene is selected from the group consisting of BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L genes.

As used herein, “cutoff value” refers to a numerical value or other representation whose value is used to arbitrate between two or more states of classification for a biological sample. In some embodiments of the invention, the cutoff value is used to distinguish positive or negative HRD status. If the HRD score is greater than the cutoff value, the HRD status is determined as positive; or if the HRD score is less than the cutoff value, the HRD status is determined as negative.

As used herein, “imbalanced chromosome arm” means copy number loss or gain of the chromosome arm. In some embodiments, an imbalanced chromosome arm refers to a chromosome arm with at least 70%, 75%, 80%, 85%, 90%, 95%, or 100% of the non-homozygous SNP loci with LOH.

As used herein, “tumor purity” is the proportion of cancer cells in a tumor sample. Tumor purity impacts the accurate assessment of molecular and genomics features as assayed with NGS approaches. In some embodiments of the disclosure, the sample has a tumor purity at least 5, 10, 15, 20, 25, 30, 35, 40, 45, 50, 55, 60, 65, 70, 75, 80, 85, 90, 95, or 100%.

As used herein, “depth” refers to the number of sequencing reads per location. “Mean depth” refers to the average number of reads across the entire sequencing region. Generally, the mean depth has an impact on the performance of the NGS assay. The higher the mean depth, the lower the variability in the variant frequency of the variant. In some embodiments of the disclosure, the mean depth of the sample across the entire sequencing region is at least 200×, 300×, 400, 500×, 600×, 700×, 800×, 900×, 1000×, 2000×, 3000×, 4000×, 5000×, 6000×, 8000×, 10000×, or 20000×.

As used herein, “coverage” refers to the depth at a given locus. “Target base coverage” refers to the percentage of the sequenced region that is sequenced at a depth above a predefined value. Target base coverage needs to specify the depth at which it is evaluated. In some embodiments, the target base coverage at 100× is 85%. That means 85% of the target sequenced bases are covered by at least 100× depth sequencing reads. In some embodiments, the target base coverage at 30×, 40×, 50×, 60×, 70×, 80×, 90×, 100×, 125×, 150×, 175×, 200×, 300×, 400×, 500×, 750×, 1000× is above 70%, 75%, 80%, 85%, 90%, or 95%.

As used herein, “subject” or “human subject” refers to those with formally diagnosed disorders, those without formally recognized disorders, those receiving medical attention, those at risk of developing the disorders, etc.

As used herein, “treat,” “treatment” and “treating” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors.

As used herein, “therapeutically effective amount” means an amount of a therapeutically active molecule needed to elicit the desired biological or clinical effect. In preferred embodiments of the disclosure, “a therapeutically effective amount” is the amount of drug needed to treat cancer patients with HRD positive.

The present disclosure is further illustrated by the following Examples, which are provided for the purpose of demonstration rather than limitation.

### EXAMPLES

### Example 1: Stability Test of Algorithms for LOH Scoring

This study was designed to evaluate the stability of the LOH score derived from different algorithms.

In silico downsampling was applied to randomly selected 260K, 150K, 100K, 50K, 40K, 30K, 20K, 10K, 9K, 8K, 7K, 6K, 5K, 4K, 3K, 2K, and 1K SNP loci from Affymetrix GeneChip Human Mapping 250K Nspl array data which was published at GEO with GEO number: GSE39130 [Wang, Birkbak, et al., 2012]. We first assigned chromosome arm information to each SNP locus in this array data and defined allele frequency ranges for homozygous, heterozygous, and loss of heterozygosity SNP loci. In silico downsampling was performed by stratified sampling at chromosome arm level to obtain designated numbers of SNPs. We sampled SNP loci in each chromosome arm independently to ensure that the number of SNP loci in each chromosome arm in the downsampled set is proportional to the original dataset. 100 bootstrap sample size was generated to evaluate the variation of LOH score of the different algorithms at each different number of SNP loci. Equation 1 considered the number of LOH SNPs, which calculated the LOH score by the ratio of the number of the loss of heterozygosity SNPs to the number of non-homozygous SNPs. In contrast, Equation 2 considered the total length of LOH SNPs, which determined the LOH score by the ratio of the total length of loss of heterozygosity SNP regions to the genome size. The analysis was performed using R (version 4.0.0).

$$LOH = \frac{number of LOH SNPs}{number of non - homozygous SNPs} \qquad ( Equation 1 )$$

$$LOH = \frac{Total length of LOH SNPs}{Genome size} \qquad ( Equation 2 )$$

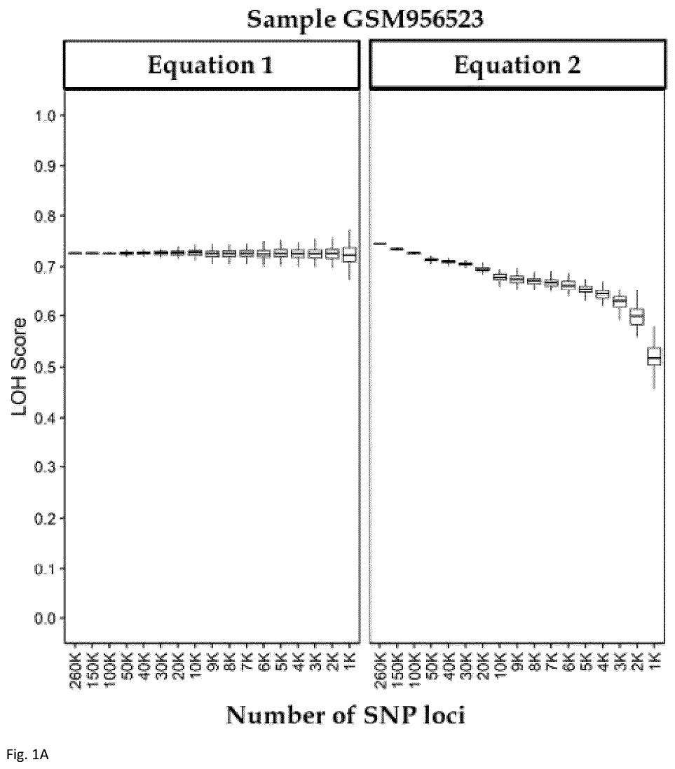

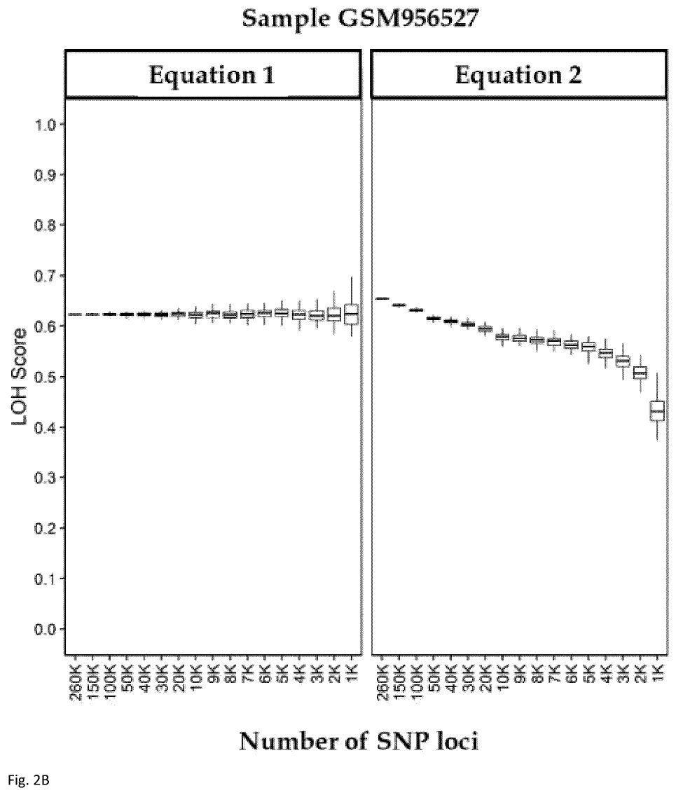

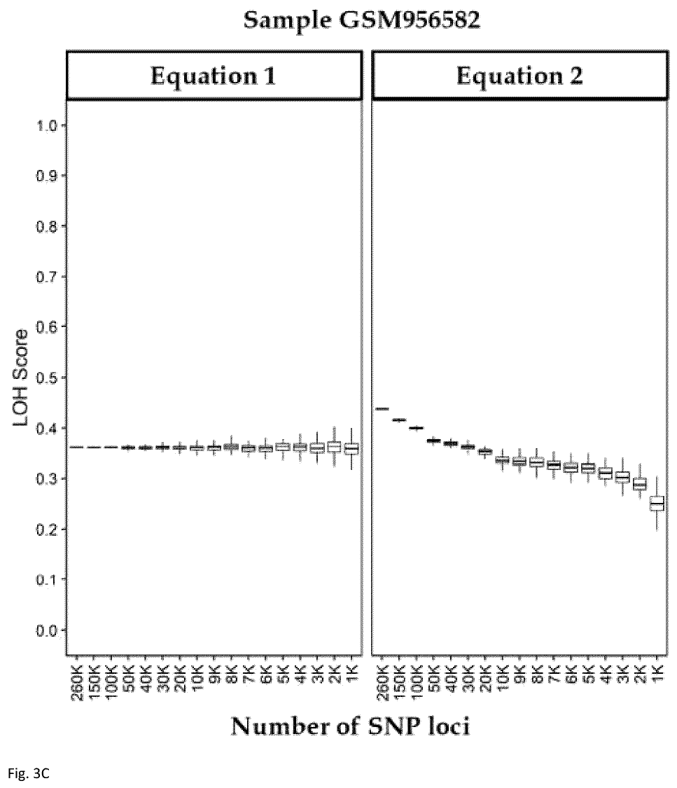

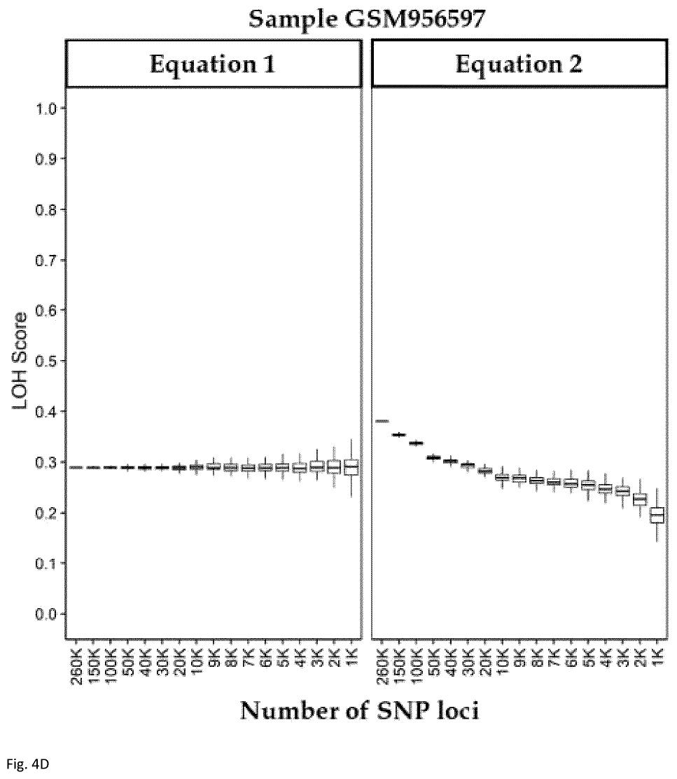

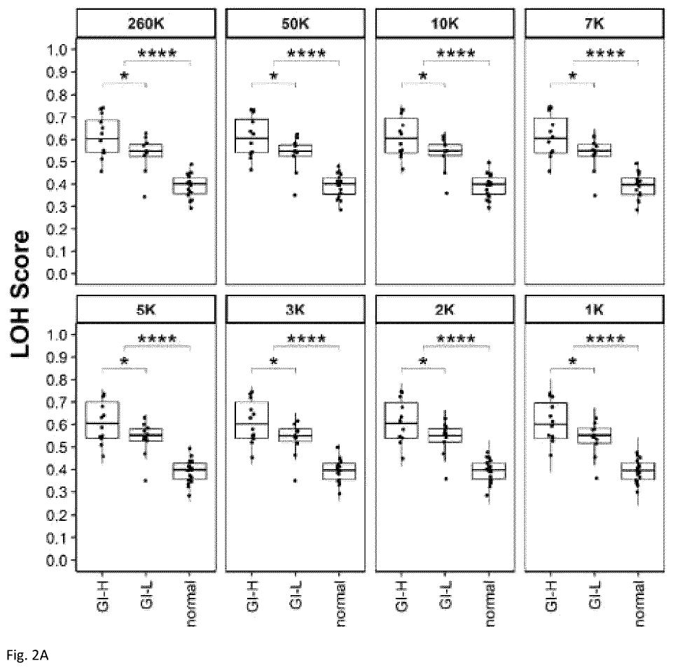

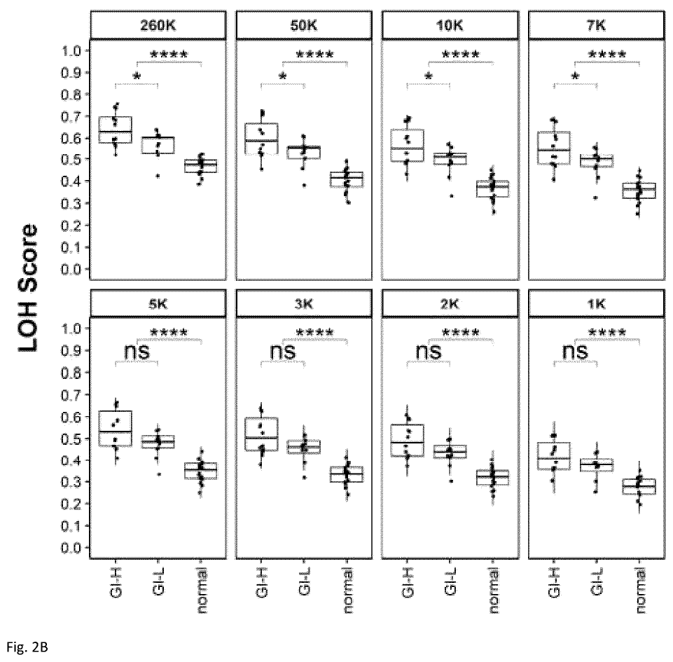

FIGS. 1A-1D show the LOH scoring results of two tumor samples (GSM956523 and GSM956527) and two normal samples (GSM956582 and GSM956597) using two different algorithms. The calculation results of using Equation 1 show a stable median LOH score at a different number of SNP loci when the LOH score derived from Equation 2 is descending as the number of SNP loci decreases.

### Example 2: Validation of LOH Scoring Algorithms

This study selects an algorithm that estimates LOH score having significant differences between tumor and normal groups at a different number of SNP loci.

All samples used in the study were analyzed in this study [Wang, Birkbak, et al., 2012]. Tumor samples with BRCA2 LOH were clustered to the high genomic instability group (GI-H), and in contrast, the low instability group (GI-L) was the tumor samples without BRCA2 LOH. Since cells with BRCA2 LOH demonstrated genomic instability and showed high sensitivity to DNA damaging agents, GI-H group in this study could potentially represent drug sensitive group, and GI-L group could potentially represent drug-resistant groups. There were 12, 11, and 18 samples in GI-H, GI-L, and normal groups, respectively. LOH score of each sample was estimated by Equation 1 and Equation 2 described in Example 1 at the number of SNP loci of 260K, 50K, 10K, 7K, 5K, 3K, 2K, and 1K. Wilcoxon signed-rank test was applied to estimate the p-value of LOH score between GI-H, GI-L, and normal samples.

We found a significant LOH score difference between 2 tumor groups: GI-H and GI-L, using Equation 1 at all different numbers of SNP loci (p-value <0.05). However, the LOH score is significantly different between two tumor groups only when the number of the SNP loci is equal to or higher than 7K by using Equation 2.

### Example 3: LOH Scoring for Samples at Different Tumor Purity Levels with or without Considering Chromosome Arm Imbalance Factor

This study is to evaluate the impact of chromosome arm imbalance when calculating LOH scores.

A cancer cell line sample (NCL-H1395) with copy number alteration was mixed with its match-normal sample to mimic different tumor purity levels. The experimental procedure includes DNA extraction, library construction and NGS sequencing are in accordance with Example 5. LOH scores of the mixed sample was estimated by three different algorithms at different tumor purity levels. The first algorithm calculated the LOH score without considering the impact of chromosome arm imbalance (Equation 1). The second and third algorithms considered the chromosome arm imbalance factor, which excluded the SNPs located on the imbalanced chromosome arms (Equation 3). The imbalanced chromosome arm is characterized by a ratio of the number of LOH SNP loci to the number of the non-homozygous SNP loci in a chromosome arm. The ratio in this example is 85%. The third algorithm further adjusted the ratio of the non-homozygous SNP loci with LOH for characterizing imbalanced chromosome arms based on different tumor purity levels.

$$LOH = \frac{\begin{matrix}number of LOH SNPs in non - imbalanced \\ chromosome arms\end{matrix}}{\begin{matrix}number of non - homozygous SNPs \\ in non - imbalanced chromosome arms\end{matrix}} \qquad ( Equation 3 )$$

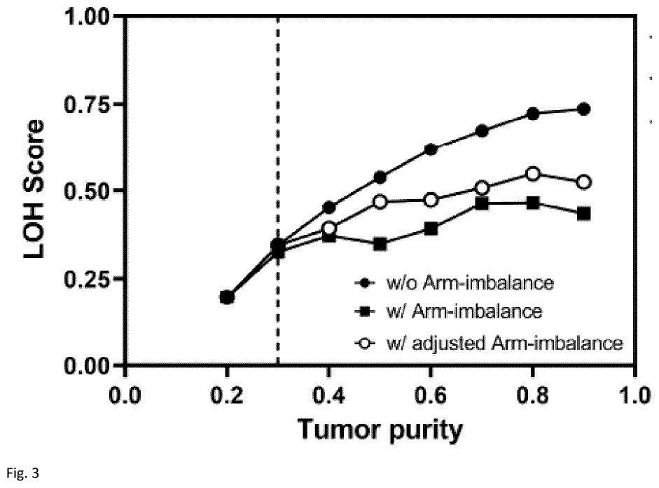

FIG. 3 shows the LOH scoring results using three different algorithms at different tumor purity levels. The LOH scores calculated by the first algorithm are increasing sharply along with the increased tumor purity. In contrast, the LOH scores calculated by the second and the third algorithms are stable when the tumor purity is larger than 30%.

TABLE 1
LOH score at different tumor purity levels considering
or ignoring chromosome arm imbalance factor

| | | | | | LOH score | | |
|---|---|---|---|---|---|---|---|
| | | Target base | | | | | w/adjusted |
| Sample | Mean | coverage at | Tumor/Normal | | w/o Arm- | w/Arm- | Arm- |
| type | depth | 100x | percentage | | imbalance | imbalance | imbalance |
| Cell | 668 | 96.51% | 90.00% | 10.00% | 0.74 | 0.44 | 0.52 |
| line | 939 | 98.35% | 80.00% | 20.00% | 0.72 | 0.47 | 0.55 |
| | 753 | 97.75% | 70.00% | 30.00% | 0.67 | 0.46 | 0.51 |
| | 661 | 97.14% | 60.00% | 40.00% | 0.62 | 0.39 | 0.47 |
| | 709 | 97.54% | 50.00% | 50.00% | 0.54 | 0.35 | 0.47 |
| | 633 | 97.20% | 40.00% | 60.00% | 0.45 | 0.37 | 0.39 |
| | 589 | 96.91% | 30.00% | 70.00% | 0.35 | 0.33 | 0.35 |
| | 695 | 97.60% | 20.00% | 80.00% | 0.20 | 0.20 | 0.20 |

### Example 4: Determining HRD Status of Cancer Samples

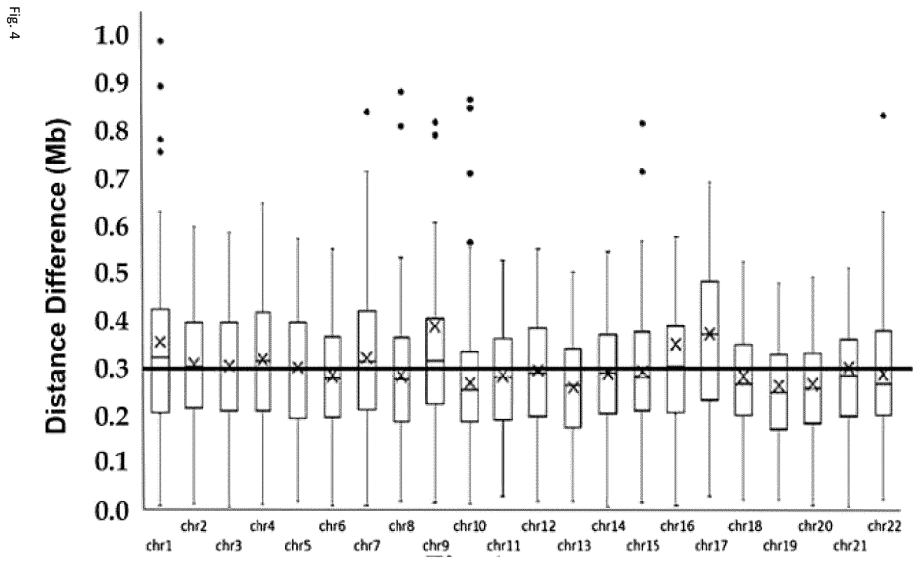

An amplicon-based NGS panel was designed targeting the coding regions of Panel A, including ARID1A, ATM, ATR, ATRX, BARD1, BRCA1, BRCA2, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L, and about 9000 SNP loci across the human genome. The mean of the intervals between the SNP loci was about 0.3 Mb in length (FIG. 4).

FFPE samples and PBMC from cancer patients were collected and assayed using the NGS panel. Genomic DNA was extracted using RecoverAll™ Total Nucleic Acid Isolation Kit (Thermo Fisher Scientific). NGS library was constructed following the user guide of CleanPlex NGS panel (Paragon Genomics, USA). Briefly, 60 ng DNA was amplified by multiplex PCR reaction using primers targeting the region designed above. After purification using magnetic beads, CP digestion, and second purification, a second PCR reaction was performed using the i5 and i7 indexing primer for Illumina following the user guide. After another purification, samples were run through capillary electrophoresis (FragmentAnalyzer, AATI). Samples that pass library qualify control (QC) are combined for sequencing on NextSeq550 (Illumina, USA) following the manufacturer's system guide and Illumina NextSeq System Denature and Dilute Libraries Guide.

Raw reads generated by the sequencer were mapped to the hg19 reference genome using BWA (version 0.7.17). SNVs and INDELs were identified using Pisces (version 5.2.5.20). VEP (Variant Effect Predictor) (version 88) was used to annotate every variant using databases from Clinvar (version 20180729) and Genome Aggregation database r2.1.1. Coverage analyses were performed by bedtools and samtools to calculate the depth at each target base and target amplicon in the panel.

Samples QC was performed to make sure that the mean sequencing depth of each sample reached 1000×.

For determining LGR and CNV, amplicons with read counts in the lowest 1st percentile and highest 0.5 percentile of all detectable amplicons and amplicons with a coefficient of variation >0.35 were removed. The remaining amplicons were normalized to correct the pool design bias. ONCOCNV (an established method for calculating copy number aberrations in amplicon sequencing data by Boeva et al., 2014) was applied for the normalization of total amplicon number, amplicon GC content, amplicon length, and technology-related biases, followed by segmenting the sample with a gene-aware model. Observed copy numbers of each gene and exon were calculated using ONCOCNV. Aberration Detection in Tumour Exome (ADTEx) software (Amarasinghe et al., 2014) was used to calculate the tumor purity of each FFPE sample. Adjusted copy numbers for each gene were calculated by adjusting for tumor purity in FFPE samples.

SNPs were determined as LOH or heterozygous according to their variant allele frequencies. The LOH score of a sample is calculated by taking the proportion of SNPs with LOH status according to Equation 3.

When there is chromosome arm imbalance detected, all SNPs on the chromosome arm are excluded from the analysis. Here, chromosome arm imbalance is detected as either a copy number gain or loss for the entire chromosome arm.

The LOH score of the samples tested are listed in Table 2.

TABLE 2
LOH score of samples tested

| | | Tumor | Mean | LOH |
|---|---|---|---|---|
| Sample ID | Cancer type | purity | depth | score |
| E01242 | Ovarian cancer | 45% | 489 | 0.28 |
| F00627 | Pancreatic cancer | 61% | 960 | 0.13 |
| ACT1925 | Lung cancer | 67% | 595 | 0.29 |
| ACT1850 | Lung cancer | 43% | 365 | 0.53 |
| E01900 | Ovarian cancer | 65% | 940 | 0.2 |
| F01519 | Ovarian cancer | 57% | 730 | 0.65 |
| AA-20-01148 | Ovarian cancer | 78% | 895 | 0.49 |
| ACT1730 | Prostate cancer | 71% | 322 | 0.22 |
| E00195 | Prostate cancer | 58% | 414 | 0.63 |
| F00044 | Prostate cancer | 40% | 415 | 0.29 |

### Example 5: LOH Distribution of Samples in Different Genotype Groups

This study was designed to evaluate the LOH score distribution of samples with different genotypes of Panel A, including ARID1A, ATM, ATR, ATRX, BARD1, BRCA1, BRCA2, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, and RAD54L.

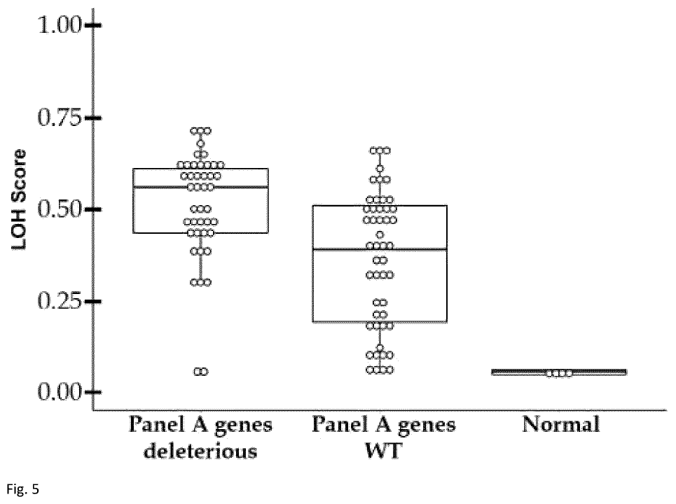

A total of 92 ovarian cancer samples and 4 normal samples were sequenced by the assay in Example 4, and the LOH score of each sample was calculated by Equation 3. Samples having pathogenic mutations or likely pathogenic mutations in genes of Panel A were grouped to Panel A genes deleterious. In contrast, the other samples having no pathogenic or likely pathogenic mutations in all genes of Panel A were considered Panel A genes WT. The distribution of the sample LOH score of each group is shown in FIG. 5.

The distribution of LOH score of samples in different groups shows that group of Panel A genes deleterious have higher LOH scores than the other groups.

1. A method for assessing homologous recombination deficiency (HRD) status in a subject, comprising:
(1) sequencing a plurality of single nucleotide polymorphism (SNP) loci of a sample from the subject, wherein there is an interval between every two neighboring SNP loci and at least 50% of the intervals are 0.01 to 1 Mb in length;
(2) identifying a number of loss of heterozygosity (LOH) SNP loci and a number of nonhomozygous SNP loci based on the sequencing result;
(3) calculating a LOH score, wherein the LOH score is a ratio of the number of LOH SNP loci to the number of non-homozygous SNP loci;
(4) identifying a HRD status based on the LOH score.

2. The method of claim 1, wherein the plurality of SNP loci is at least 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, or 300000 in number.

3. The method of claim 1, wherein the plurality of SNP loci is 2500 to 250000 in number.

4. The method of claim 1, wherein the plurality of SNP loci is 3000 to 60000 in number.

5. The method of claim 1, wherein the plurality of SNP loci is 6000 to 11000 in number.

6. The method of claim 1, wherein the plurality of SNP loci are in at least 2 pairs of chromosomes.

7. The method of claim 1, wherein the plurality of SNP loci are in 22 pairs of chromosomes.

8. The method of claim 1, wherein a mean of the intervals is 0.01 to 3 Mb, 0.02 to 2 Mb, 0.03 to 1 Mb, 0.06 to 1 Mb, 0.1 to 1 Mb, 0.06 to 0.6 Mb, 0.1 to 0.5 Mb, or 0.2 to 0.4 Mb in length.

9. The method of claim 1, wherein Step (3) further comprises adjusting the LOH score through eliminating imbalanced chromosome arms.

10. The method of claim 9, wherein the LOH score is a ratio of number of LOH SNP loci in nonimbalanced chromosome arms to number of the non-homozygous SNP loci in non-imbalanced chromosome arms.

11. The method of claim 9, wherein the imbalanced chromosome arm is characterized by a predetermined ratio of number of LOH SNP loci to number of the non-homozygous SNP loci in a chromosome arm, wherein the predetermined ratio is at least 70%, 75%, 80%, 85%, 90%, 95%, 98%, or 100%.

12. The method of claim 11, wherein the predetermined ratio is further adjusted based on a tumor purity of the sample.

13. The method of claim 12, wherein the tumor purity is 30% to 95%.

14. The method of claim 12, wherein the tumor purity is 30% to 70%.

15. The method of claim 1, wherein the HRD status is identified as positive or negative.

16. The method of claim 1, wherein a cutoff value of the LOH score for identifying the HRD status is 0.3, 0.35, 0.4, 0.45, 0.5, 0.55, or 0.6.

17. A method for assessing HRD status in a subject, comprising:
(1) sequencing a gene including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof of a sample from the subject;
(2) determining whether the gene harbors an alteration; and
(3) identifying a HRD status based on the determination result.

18. The method of claim 17, wherein the HRD status is positive when the gene harbors an alteration.

19. The method of claim 17, wherein the HRD status is negative when none of the gene harbors an alteration.

20. The method of claim 17, wherein the alteration is a germline alteration or a somatic alteration.

21. The method of claim 17, wherein the alteration is selected from the group consisting of single nucleotide variant (SNV), insertion, deletion, amplification, gene fusion, and rearrangement.

22. The method of claim 17, wherein the alteration is selected from the group consisting of SNV, small insertions and deletion (INDEL), large genomic rearrangement (LGR), and copy number variation (CNV).

23. A method for assessing HRD status in a subject, comprising:
(1) assaying an alteration of a gene in a sample from the subject, comprising:
(1a) sequencing the gene comprising BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof of the sample;
(1b) determining whether the gene harbors an alteration;
(2) calculating a HRD score of the sample, comprising:
(2a) sequencing a plurality of single nucleotide polymorphism (SNP) loci of the sample;
(2b) calculating the HRD score of chromosomal aberration; and
(3) identifying a HRD status based on the result of Step (1b), Step (2b) or the both thereof.

24. A method for assessing HRD status in a subject, comprising:
(1) assaying an alteration of a gene in a sample from the subject, comprising:
(1a) sequencing a HRR-associated gene; and
(1b) determining whether the HRR-associated gene harbors an alteration;
(2) calculating a LOH score of the sample, comprising:
(2a) sequencing a plurality of SNP loci of the sample, wherein there is an interval between every two neighboring SNP loci and at least 50% of the interval is 0.01 to 1 Mb in length; and
(2b) calculating a ratio of a number of LOH SNP loci to a number of non-homozygous SNP loci; and
(3) identifying a HRD status based on the result of Step (1b), Step (2b) or the both thereof.

25. The method of claim 23, wherein the HRD status is positive when the gene in Step (1) harbors an alteration or the score in Step (2) is greater than a cutoff value.

26. The method of claim 1, further comprises a step of identifying a treatment based on the HRD status for the subject.

27. The method of claim 26, further comprises a step of administering a therapeutically effective amount of the treatment to the subject.

28. The method of claim 26, wherein the treatment is selected from the group consisting of DNA damaging agent, anthracycline, topoisomerase I inhibitor, radiation, PARP inhibitor and any combination thereof.

29. The method of claim 28, wherein the PARP inhibitor is selected from the group consisting of olaparib, niraparib, rucaparib, and talazoparib.

30. The method of claim 1, wherein the method for assessing HRD status in a sample is implemented on a next-generation sequencing (NGS) computing platform.

31. The method of claim 1, wherein the sample is sequenced by NGS assay.

32. The method of claim 1, wherein the sample is originated from cell line, biopsy, primary tissue, frozen tissue, formalin-fixed paraffin-embedded (FFPE), liquid biopsy, blood, serum, plasma, buffy coat, body fluid, visceral fluid, ascites, paracentesis, cerebrospinal fluid, saliva, urine, tears, seminal fluid, vaginal fluid, aspirate, lavage, buccal swab, circulating tumor cell (CTC), cell-free DNA (cfDNA), circulating tumor DNA (ctDNA), DNA, RNA, nucleic acid, purified nucleic acid, purified DNA, or purified RNA.

33. The method of claim 1, wherein the subject is a human.

34. The method of claim 1, wherein the subject is a cancer patient.

35. The method of claim 1, wherein a tumor purity of the sample is at least 5, 10, 15, 20, 25, 30, 35, 40, 45, 50, 55, 60, 65, 70, 75, 80, 85, 90, 95, or 100%.

36. The method of claim 1, further comprises a step of outputting the HRD status to an electronic storage medium or a display.

37. A system for assessing HRD status, comprising:
a data storage device storing instructions for determining characteristics of a HRD status and a processor configured to execute the instructions to perform a method comprising:
(1) sequencing a plurality of single nucleotide polymorphism (SNP) lociof a sample from a subject, wherein there is an interval between every two neighboring SNP loci and at least 50% of the intervals are 0.01 to 1 Mb in length;
(2) identifying a number of loss of heterozygosity (LOH) SNP loci and a number of nonhomozygous SNP loci by based on the sequencing result;
(3) calculating a loss of heterozygosity (LOH) score, wherein the LOH score is a ratio of the number of LOH SNP loci to the number of non-homozygous SNP loci; and
(4) identifying the HRD status based on the LOH score.

38. A system for assessing HRD status, comprising:
a data storage device storing instructions for determining characteristics of a HRD status and a
processor configured to execute the instructions to perform a method comprising:
(1) sequencing a gene including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof of a sample from a subject;
(2) determining whether the gene harbors an alteration; and
(3) identifying a HRD status based on the determination result.

39. A system for assessing HRD status, comprising:
a data storage device storing instructions for determining characteristics of a HRD status and a
processor configured to execute the instructions to perform a method comprising:
(1) assaying an alteration of a gene in a sample from a subject, comprising:
(1a) sequencing a gene including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof of the sample; and
(1b) determining whether the gene harbors an alteration;
(2) calculating a HRD score of the sample, comprising:
(2a) sequencing a plurality of single nucleotide polymorphism (SNP) loci of the human sample; and
(2b) calculating the HRD score of a chromosomal aberration; and
(3) identifying the HRD status based on the result of Step (1b), Step (2b) or the both thereof.

40. A system for assessing a HRD status, comprising:
a data storage device storing instructions for determining characteristics of a HRD status and a processor configured to execute the instructions to perform a method comprising:
(1) assaying an alteration of a gene of a sample from a subject, comprising:
(1a) sequencing a HRR-associated gene of the sample; and
(1b) determining whether the HRR-associated gene harbors an alteration;
(2) calculating a LOH score of the sample, comprising:
(2a) sequencing a plurality of SNP loci of the sample, wherein there is an interval between every two neighboring SNP loci and at least 50% of the interval are 0.01 to 1 Mb in length; and
(2b) calculating a ratio of a number of LOH SNP loci to a number of non-homozygous SNP loci; and
(3) identifying the HRD status based on the result of Step (1b), Step (2b) or the both thereof.

41. The system of claim 37, further comprises a step of identifying a treatment based on the HRD status for the subject.

42. The system of claim 41, further comprises a step of administering a therapeutically effective amount of a treatment to the subject.

43. A kit for assessing HRD status of a subject, comprising:
a reagent, comprising:
a set of oligonucleotides targeting a plurality of SNP loci, wherein there is an interval between every two neighboring SNP loci and at least 50% of the interval are 0.01 to 1 Mb in length; and
a computer program, comprising:
instructions to calculate a LOH score, wherein the LOH score is a ratio of a number of LOH SNP loci to a number of non-homozygous SNP loci; and
instructions to identify a HRD status.

44. A kit for assessing HRD status of a subject, comprising:
a reagent, comprising:
a set of oligonucleotides targeting a gene including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof of a sample from the subject; and
a computer program, comprising:
instructions to determine whether the gene harbors an alteration; and
instructions to identify the HRD status.

45. A kit for assessing HRD status in a subject, comprising:
a reagent, comprising:
a set of oligonucleotides for targeting a plurality of SNP loci, wherein there is an interval between every two neighboring SNP loci and at least 50% of the interval are 0.01 to 1 Mb in length; and
a set of oligonucleotides for targeting a HRR-associated gene;
a computer program, comprising:
instructions to calculate a LOH score, wherein the LOH score is a ratio of a number of LOH SNP loci to a number of non-homozygous SNP loci;
instructions to determine whether the HRR-associated gene harbors an alteration; and
instructions to identify a HRD status.

46. A kit for assessing HRD status in a subject, comprising:
a reagent, comprising:
a set of oligonucleotides for targeting a plurality of SNP loci of a sample from the subject; and
a set of oligonucleotides for targeting a gene including BRCA1, BRCA2, ARID1A, ATM, ATR, ATRX, BARD1, BRIP1, CDK12, CHEK1, CHEK2, FANCA, FANCL, FANCM, HDAC2, NBN, PALB2, PPP2R2A, PTEN, RAD51, RAD51B, RAD51C, RAD51D, RAD54L or any combination thereof;
a computer program, comprising:
instructions to calculate a HRD score of chromosomal aberration;
instructions to determine whether the gene harbors an alteration; and
instructions to identify a HRD status.

47. The kit of claim 43, wherein the HRD status is positive when the LOH score is greater than a cutoff value.

48. The kit of claim 44, wherein the HRD status is positive when the gene harbors an alteration.

49. The kit of claim 45, wherein the HRD status is positive when the LOH score is greater than a cutoff value or the HRR-associated gene harbors an alteration.

50. The kit of claim 46, wherein the HRD status is positive when the HRD score is greater than a cutoff value or the genes harbors an alteration.

51. The kit of claim 43, wherein the computer program further comprises instructions to identify a treatment based on the HRD status for the subject.
